(19) United States
(12) Patent Application Publication
(10) Pub. No.: US 2005/0240248 A1
(43) Pub. Date: Oct. 27, 2005

(54) High pressure tanning booth

(76) Inventor: Ralph Venuto SR., Blackwood, NJ (US)

Correspondence Address: BLANK ROME LLP, 600 NEW HAMPSHIRE AVENUE, N.W., WASHINGTON, DC, 20037, US

(21) Appl. No.: 10/830,442

(22) Filed: Apr. 23, 2004

(51) Int. Cl. A61N 5/06

(52) U.S. Cl. 607/91; 607/94

(57) ABSTRACT

A tanning booth has high density tanning lamps and a ventilation system capable of dissipating the large amounts of heat generated by the high density tanning lamps, as well as the heat generated by a ballast that is used with the lamp. Multiple lamp units are positioned about the tanning booth. Each lamp unit includes a main body that retains a lamp housing and a blower. The lamp housing has one or more lamps, reflector panels, and a first glass cover. An air passageway is created by a second glass cover spaced parallel and apart from the first glass panel. The blower draws air into the housing from a first set of ventilation openings in the tanning booth wall, through the air passageway, and out a second set of ventilation openings in the tanning booth wall. In an alternative embodiment, the first glass panel can be removed so that air blows directly across the lamp.

### BACKGROUND OF THE INVENTION

1. Field of the Invention

The present invention relates to a tanning booth having high-pressure lamps. More particularly, the present invention relates to a tanning booth having a ventilation system that efficiently removes heat produced by the high-pressure lamps.

2. Background of the Related Art

Tanning booths have been developed with tanning lamps so that a user can obtain and maintain a tan all year round, regardless of weather conditions. Tanning booth technology continues to improve. However, it is a goal to provide a quality tan in a reduced amount of time in order to be convenient for users as well as to increase the value of the tanning booth by being able to accommodate a greater number of users during the day.

### SUMMARY OF THE INVENTION

In view of the foregoing, one object of the invention is to provide a tanning booth that is able to minimize the time needed to provide a tan. Another objection of the invention is to provide a tanning booth with an efficient ventilation system that dissipates substantial amounts of heat generated by tanning lamps, and especially high-pressure tanning lamps. It is yet another object of the invention to provide a tanning booth in which each tanning lamp is provided with its own ventilation system that dissipates the heat generated by the tanning lamp and the electronic components that accompany the tanning lamp.

In accordance with these and other objects, the present invention is a tanning booth having high density tanning lamps and a ventilation system capable of dissipating the large amounts of heat generated by the high density tanning lamps, as well as a ballast that is used with the lamp. Multiple lamp units are positioned about the tanning booth. Each lamp unit includes a main body that retains a lamp housing and a blower. The lamp housing has one or more lamps, reflector panels, and a first glass cover. An air passageway is created by a second glass cover spaced parallel and apart from the first glass panel. The blower draws air into the housing from a first set of ventilation openings in the tanning booth wall, through the air passageway, and out a second set of ventilation openings in the tanning booth wall. In an alternative embodiment, the first glass panel can be removed so that air blows directly across the lamp.

These and other objects of the invention, as well as many of the intended advantages thereof, will become more readily apparent when reference is made to the following description, taken in conjunction with the accompanying drawings.

### BRIEF DESCRIPTION OF THE FIGURES

FIG. 1 is a top view of the tanning booth in accordance with the preferred embodiment of the invention.

FIG. 2 is a front perspective view of the tanning booth taken along lines A-A of FIG. 1.

FIG. 3 is a front view of the lamp unit used in the tanning booth.

FIG. 4 is a rear view of the lamp unit used in the tanning booth.

FIG. 5 is a side view of the lamp unit taken along lines B-B of FIG. 2.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in similar manner to accomplish a similar purpose.

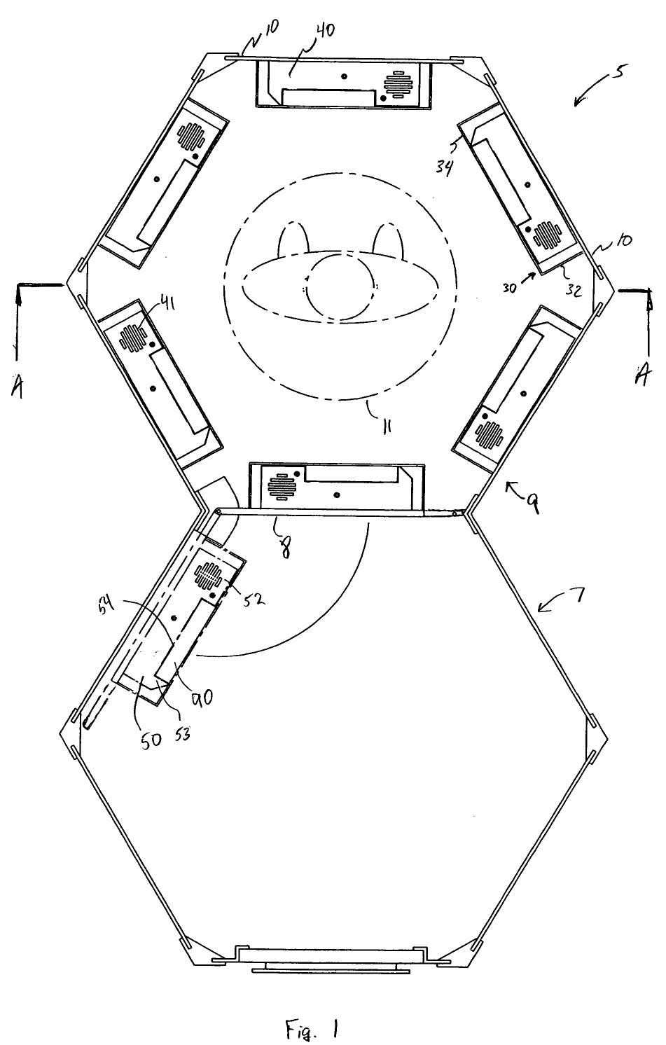

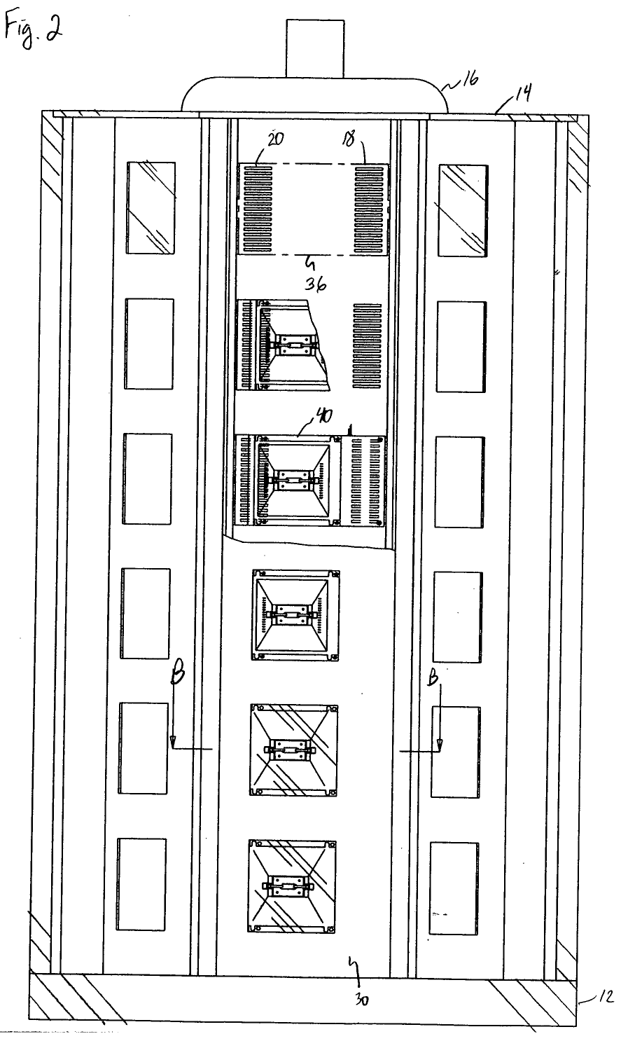

Turning to the drawings, FIGS. 1 and 2 show a tanning booth 5 in accordance with the preferred embodiment of the invention. The booth 5 has rectangular wall panels 10, a floor frame 12, a ceiling frame 14, and a fan 16. The wall panels 10 are preferably positioned to form two hexagonal enclosures 7, 9 that form a figure eight shape with a door 8 between the two enclosures. The front enclosure 7 is a dressing room and the rear enclosure 9 is a tanning area. The booth 5 can be constructed, for instance, in accordance with U.S. Pat. No. 6,673,092 to Venuto, which is incorporated herein by reference. Each wall 10 and optionally the door 8 have a series of first and second sets of ventilation openings 18, 20 that communicate air between the exterior and the interior of the enclosure 9.

Lamp units 40 are arranged on each wall 10, positioned within a housing panel 30. The housing panel 30 has two side panels 32 and a front panel 34 that essentially form a U-shape. The front panel has openings 36 that each receive a respective lamp unit 40. Each side panel 32 has an end with a flange 38 (FIG. 5) that is secured to the wall 10 of the enclosure 9 by one or more fasteners. The lamp units 40 are secured to the housing panel 30 within the openings 36 so that the front of the lamp unit 40 is substantially flush with the front 34 of the housing panel 30 at the interior of the enclosure 9. The housing panel 30 preferably extends the entire height of the booth enclosure 9 from the floor 12 to the ceiling 14, so that heated air contained in the panel 30 does not escape into the interior of the booth 9.

Turning to FIGS. 3-5, the lamp units 40 are shown in greater detail. Each lamp unit 40 has a main housing or body 50, a ventilation system 80, one or more lamps or bulbs 42, and a lamp housing 60. Preferably, the top row of lamp units 40 each have two lamps 42, so that there is a greater concentration of UV radiation at the user's face. The remaining lamp units 40 have a single lamp 42. Since there are six walls 10 in the enclosure 9, and each wall 10 holds six lamp units 40, there are a total of forty-two bulbs 42.

The lamp housing 60 retains the lamps 42, and includes one or more pairs of lamp receptacles 62, reflector panels 64, a rear reflector panel 69, and a glass cover 68. The side reflector panels 64 have ventilation openings 66 that permit heated air to be vented to the outside of the lamp housing 60, into the space between the lamp unit 40 and the enclosure wall 10. The heated air passes out of the enclosure 9 through the second set of ventilation openings 20 in the wall 10. The lamp receptacles 62 are mounted to a bracket 63 and each pair of lamp receptacles 62 receives a lamp 42 that clips into the lamp receptacles 62. Preferably, the bracket 63 can receive up to three pairs of lamp receptacles 62. The lamp 42 is located at the center of the reflector panels 64, which are positioned to direct UV radiation into the tanning booth enclosure 9.

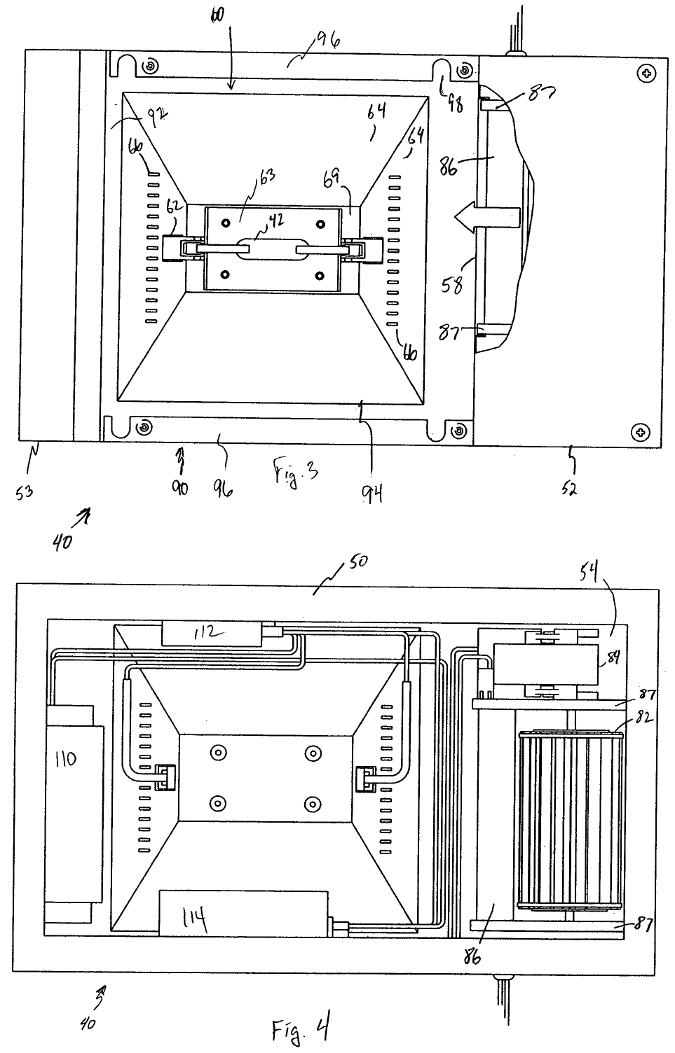

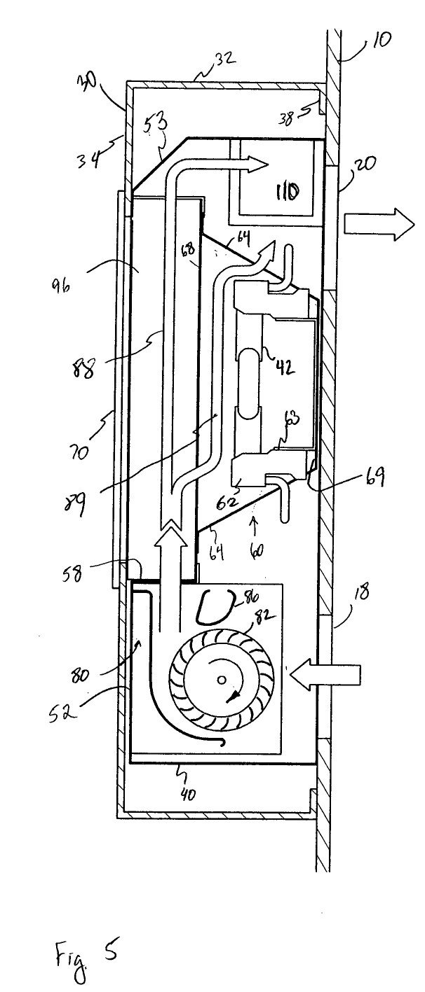

The bulbs 42 used in the preferred embodiment are commonly referred to as high-pressure bulbs 42, and are manufactured by Aegis, Inc. of St. Petersburg, Fla. The high-pressure bulbs 42 impart a large amount of UV radiation, so that the user can achieve a tan in a relatively short period of time. In accordance with the preferred embodiment, a user can obtain a tan in 12-18 minutes. However, the high-pressure bulbs 42, as well as the ballasts 110 (FIG. 4) needed to operate them, emit substantial amounts of heat.

A ventilation system 80 is provided to dissipate the heat generated by the high-pressure bulbs 42 and the ballast 110. In accordance with the preferred embodiment, the ventilation system 80 includes a fan or blower 82, a motor 84 for driving the blower 82, a baffle 86, and an air passageway 88. The main body 50 has a blower housing portion 52 with a mounting platform 54. The blower 82, motor 84 and baffle 86 are connected to the mounting platform 54. A slot 58 is located between the mounting platform 54 and the lamp receiving portion 90 to permit air to communicate therebetween.

The lamp unit 40 is a single piece of metal having two portions, the main body portion 50, and a lamp receiving portion 90, as best shown in FIG. 1. A blower housing portion 52 is located at one end of the main body 50, and a conduit 53 is located at the opposite end of the main body 50. The mid-section 54 of the body 50 is narrower than the two ends 52, 53, to receive the lamp housing 60 and so that an air passageway 88 can be created in front of the lamp housing 60. The lamp receiving portion 90 is folded back at the front wall of the blower housing portion 52 (FIG. 5), so that it is continuous with the main body 50. Notches 98 are provided to allow a tool, such as a screwdriver, to secure a fastener to connect the lamp receiving portion 90 to the main body 50 during assembly of the lamp unit 40. Though the lamp unit 40 is a single piece, the fastener ensures that the lamp receiving portion 90 remains closely positioned to the main body 50.

The lamp receiving portion 90 includes a frame 92 having a large central opening 94. The top and bottom of the frame 92 are two arms 96 that extend outwardly from the blower housing 52. The lamp housing 60 is secured to the frame 92, within the opening 94. Each of the two arms 96 are folded to form a U-shaped track. A protective glass 70 is connected to the arms 96 or can be slidably received in the arms 96. The glass 70 preferably is substantially flush with the housing panel 30 at the interior of the booth 9. The air passageway 88 is the space between the outer glass 70 and the inner glass 68 and the two arms 96. The air passageway 88 extends the entire width of the lamp housing 60, from the slot 58 to the conduit 53.

Accordingly, the fan 82 blows air through the air passageway 88 to dissipate the heated air located in the passageway 88. The air passageway 88 operates as a barrier between the user and the heated air in the lamp housing 60. The duct 53 is beveled (FIG. 5) so that, as the air exits the passageway 88, the air is directed to the rear of the lamp unit 40 and out the second ventilation openings 20 in the wall 10 of the tanning booth enclosure 9. The air flowing through the conduit 53 also circulates about the rear of the lamp housing 60, removes any heated air from behind the lamp housing 60 that escaped through vents 66, and forces it out through the second ventilation openings 20. Air may also be forced into one set of the vents 66, across the lamp 42, and out through the other set of vents 66.

The glass 70 keeps the heated air within the lamp unit 40 so that it does not heat the interior of the booth 9. The glass 70 also creates the air passageway 88 to allow the heated air to be dissipated by the fan 82. In addition, the glass 70 protects the user from injury by preventing the user from inadvertently coming into contact with the bulb 42. The glass 70 can be coupled to the front 34 of the housing panel 30, or to the track 96, at the interior of the enclosure 9. Preferably, however, one side of the front of the track 96 extends slightly beyond the conduit 53, so that the glass 70 can be slidably removed from the inside of the enclosure 9.

However, a plastic guide can optionally be provided along the length of the arms 96, with a slot that receives the glass. The plastic guide can, for instance, be affixed within the notches 98. The first glass 68 is preferably fastened to the lamp housing 60 or the inside portion of the track 96. Thus, both glass covers 68, 70 lock in place and can be easily removed and replaced so that the bulb 42 can be replaced from the inside of the booth 9 when it has burned out.

As best shown in FIG. 2, the lamp unit 40 is positioned so that the fan 82 is aligned with the first set of ventilation openings 18 in the wall 10 of the enclosure. The ventilation system 80 directly dissipates heated air produced by the lamp 42 by blowing cooler air through the lamp unit 40. The fan 82 draws air in from the exterior of the enclosure 9 through the first set of ventilation openings 18. The baffle 86 primarily directs the air flow into the air passage 88, forcing cool air into the air passage 88 and dissipating heated air. The heated air is forced out of the air passageway 88 and out of the enclosure 9 through the second set of ventilation openings 20 in the enclosure wall 10. Thus, the blower 82 directs air in one side of the lamp unit 40, through the air passageway 88, and out the other side of the lamp unit 40. The baffle 86 also directs some air behind the lamp housing 60 to dissipate heated air escaping through vents 66.

In an alternative embodiment, the first glass cover 68 need not be provided, so that the front of the lamp housing 60 is open. Accordingly, a second air passageway 89 is created (in addition to the first air passageway 88), whereby the air flows into the lamp housing 60, past the lamp 42, through the vents 66, and out through the second ventilation openings 20. In addition, vents 41 (FIG. 1) can also be provided to permit the blower to draw air into the lamp unit 40.

The first and second set of ventilation openings 18, 20 are spaced apart so the fan 82 does not draw in, through the first ventilation openings 18, the heated air that is simultaneously being exhausted out of the second ventilation openings 20. Baffles can optionally be fitted to the enclosure about the output ventilation openings 20 to direct the heated air away (vertically and/or horizontally) from the input ventilation openings 18.

The blower 82 is preferably surrounded by partitions 87. The partitions 87 can optionally extend to the ventilation opening 18 of the wall 10 of the enclosure 9, so that the blower 82 only draws in air from the exterior of the booth 9 through the first ventilation openings 18. The partitions 87 reduce the amount of heated air that the blower 82 draws in from behind the lamp housing 60 that may be heated by the lamp 42, or heated air that passes out of the lamp housing 60 through vents 66. Instead, any heated air positioned behind the lamp housing 60 will pass out of the second ventilation openings 20 in the wall 10 of the enclosure. In addition, the air being exhausted from the lamp housing 60 by the blower 82 operates as a suction to draw heated air from behind the lamp housing 60 out of the enclosure through the second ventilation openings 20.

As further shown in FIGS. 4 and 5, various electronic components are also housed within the lamp unit 40, including a 500 Watt ballast 110, ignitor 112, and a 40 μF capacitor 114, to provide power to the lamps 42. In accordance with the preferred embodiment, the ballast 110 is positioned in the main body 50 at the conduit 53. Air coming out of the air passage 88 passes the ballast 110, thereby cooling the ballast 110. Since the preferred ballast 110 has a winding temperature of 130° C. (266° F.), it is important that the ballast 110 be effectively cooled.

Preferably, each lamp unit 40 is provided with its own blower 82 to maximize the dissipation of the heated air produced by the lamp 42 and the ballast 110. Each lamp unit 40 only accommodates up to three lamps 42, so that the unit 40 does not generate more heat than can be dissipated by the blower 82. The effectiveness of each blower 82 is maximized since a blower 82 is associated with a limited number of lamps 82 and a ballast 110. The blowers 82 are dispersed throughout the booth 9 in accordance with the disbursement of the sources of heat (i.e., the lamp 42 and the ballast 110). However, a central blower 82 can be provided and air directed to the lamp units 40 by a duct.

The number and positioning of lamps is exemplary only, and is not intended to be limiting; the optimal number may be less or more than forty-two. In addition, the tanning booth 5 need not have two rooms 7, 9. The tanning booth enclosure 9 can be provided without the use of a dressing room enclosure 7. The enclosure also need not fully surround or enclose the user.

A fan 16 is also preferably positioned in the ceiling 14 of the enclosure 9, and the floor and floor frame 12 of the enclosure 9 have openings that permit air to be drawn into the interior of the booth. The floor has a perforated hole pattern 11, and the floor frame 12 has side vents. The hole pattern 11 and side vents permit the fan 16 to draw air into the booth 9 from the outside of the booth 9 for effective cooling of the user. The pattern 11 is centered in the hexagonal room. The fan 16 draws air through the tanning booth room 9 through the floor hole pattern 11 and the side vents in the floor frame 12.

The fan 16 and floor hole pattern 11 provide sufficient cooling to the user due to the heat generated by the tanning lamps. The increased number and output of the lamps 42 require adequate cooling to ensure the comfort and safety of the user. The fan 16 and floor hole pattern 11 enable a tan time of between about 12 minutes (for people with sensitive skin that burn easily) and 18 minutes (for people with dark skin that burn minimally) without danger to the user. However, the actual tan time also depends upon other conditions, such as the moisture condition of the skin. At least three inches of ventilation space should be provided all around the booth 9 to ensure adequate ventilation of the tanning room by blower 82 and fan 16.

The tanning booth 9, including the wall panels 10, floor frame 12, floor 11, and ceiling frame 14, are constructed of steel and the booth 5 is easy to assemble. The configuration and design of the tanning booth 5 establishes a safe distance from the user to the tanning lamps 42, but at the same time maintains maximum tanning effect.

The lamp power is 300-500 Watts, 96 Watts of UVA radiation flux and 25 Watts of UVB radiation flux, so that a tan time of 12 to 18 minutes is achieved. The lamps 42 each have a voltage of 230 Volts, a current of 4.0 amperes, and a lamp voltage of 135 Volts. The preferred ballast 110 has a 60 Hz frequency, a current of 3.9 amperes, a voltage of 220 Volts, and a winding temperature of 130° C. (266° F.) with a delta temperature of 52K. The ignitor 112 has a voltage of 220 Volts, power of 400 Watts, and a current of 5.0 amperes. The ballast 110 and ignitor 112 are made by Cosmedico Light, Inc. of Weymouth, Mass.

As a result, the blower must be able to dissipate a substantial amount of heat generated by the lamp 42 and the ballast 110 in order to maintain a comfortable ambient temperature inside the booth 9 of less than about ninety degrees in the tanning booth. It should be recognized that the number and output of the lamps is exemplary only, and is not intended to be limiting; the optimal number may be less or more than the number provided.

Though the lamp unit 40 is designed to be secured to a wall 10 having ventilation openings 18, 20, other variations and uses are possible. For instance, the lamp unit 40 can be mounted in openings in the wall 10, so that a holder 30 is not needed. The lamp unit 40 can have a back plate with ventilation openings so that it is a self-contained unit that can be mounted directly in a wall opening.

The foregoing description and drawings should be considered as illustrative only of the principles of the invention. The invention may be configured in a variety of shapes and sizes and is not intended to be limited by the preferred embodiment. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A tanning booth enclosure comprising:
at least one wall defining an interior of the enclosure, said at least one wall having a first ventilation opening and a second ventilation opening spaced apart from said first ventilation opening;
a housing panel secured to said at least one wall at the interior of the enclosure, said housing panel having at least one opening; and,
a lamp unit secured to said housing panel about the at least one opening, said lamp unit including a lamp, and a blower in flow communication with said first ventilation opening in said at least one wall and positioned to direct air through said first ventilation opening, through said lamp unit, and out the second ventilation opening in said at least one wall.

2. The tanning booth enclosure of claim 1, wherein said lamp comprises a tanning lamp.

3. The tanning booth enclosure of claim 1, said lamp unit further comprising reflector panels located about said lamp.

4. The tanning booth enclosure of claim 1, wherein said lamp unit consists of a single lamp.

5. The tanning booth enclosure of claim 1, wherein said lamp comprises a high-pressure tanning lamp.

6. The tanning booth enclosure of claim 1, wherein said lamp comprises a high-density tanning lamp.

7. The tanning booth enclosure of claim 1, wherein said blower directs air directly past said lamp.

8. The tanning booth enclosure of claim 1, whereby said blower dissipates heat generated by said lamp.

9. The tanning booth enclosure of claim 1, further comprising an electrical component located inside said lamp unit and connected to said lamp, whereby said blower dissipates heat generated by said electrical component.

10. A tanning booth enclosure comprising:
at least one wall defining an interior of the enclosure, said at least one wall having a first ventilation opening and a second ventilation opening spaced apart from said first ventilation opening; and,
a lamp unit secured to said at least one wall and including a lamp, and a blower in flow communication with said first ventilation opening in said at least one wall and positioned to direct air through said first ventilation opening, through said lamp unit, and out the second ventilation opening in said at least one wall.

11. The tanning booth enclosure of claim 10, wherein said lamp comprises a tanning lamp.

12. The tanning booth enclosure of claim 10, said lamp unit further comprising reflector panels located about said lamp.

13. The tanning booth enclosure of claim 10, wherein said lamp unit consists of a single lamp.

14. The tanning booth enclosure of claim 10, wherein said lamp comprises a high-pressure tanning lamp.

15. The tanning booth enclosure of claim 10, wherein said lamp comprises a high-density tanning lamp.

16. The tanning booth enclosure of claim 10, further comprising an electrical component located inside said lamp unit and connected to said lamp, whereby said blower dissipates heat generated by said electrical component.

17. A lamp unit comprising:
a body having a blower housing portion and a lamp housing portion containing a lamp, and
a blower located inside said body at the blower housing portion, said blower drawing air into said body, creating an air flow through said lamp unit, and exhausting the air flow from said body to dissipate heat generated by the lamp.

18. The lamp unit of claim 17, wherein said body is substantially rectangular.

19. The lamp unit of claim 17, further comprising an electrical component located inside said body and connected to said lamp, whereby said blower dissipates heat generated by said electrical component.
